(19) United States
(12) Patent Application Publication
(10) Pub. No.: US 2013/0164334 A1
(43) Pub. Date: Jun. 27, 2013

(54) Dual Element Odor Control In Personal Care Products

(71) Applicants: Roger Bradshaw Quincy, III, Cumming, GA (US); Bhalchandra Madhav Karandikar, Beaverton, OR (US); John Gavin MacDonald, Decatur, GA (US); Teuta Elshani, Woodstock, GA (US)

(72) Inventors: Roger Bradshaw Quincy, III, Cumming, GA (US); Bhalchandra Madhav Karandikar, Beaverton, OR (US); John Gavin MacDonald, Decatur, GA (US); Teuta Elshani, Woodstock, GA (US)

(21) Appl. No.: 13/336,168

(22) Filed: Dec. 23, 2011

(51) Int. Cl. A61L 15/46; A01P 1/00; A61L 9/014; B82Y 30/00

(52) U.S. Cl. 424/400; 424/76.9; 604/359; 977/773; 977/902

(57) ABSTRACT

An odor control layer for personal care products includes a first portion with a formulation of activated carbon and a second portion with a formulation of nanoparticle silver. The layer can be placed in a personal care product including diapers, training pants, absorbent underpants, adult incontinence products, or feminine hygiene products. The personal care product can include a liquid-permeable bodyside liner, a liquid-impermeable outer cover affixed to the bodyside liner, and an absorbent core disposed between the bodyside liner and the outer cover.

### BACKGROUND

The present disclosure concerns processes and products for the alleviation and control of odors in personal care products.

Disposable personal care products perform a needed function in today's busy society, freeing caregivers and users from the chore of washing reusable products and allowing for the quick and easy disposal of body wastes. As leakage issues have been reduced because of improved designs, the control of odors has become more important to the consumer. This is a particular concern to users of incontinence and feminine hygiene products.

Odor is often used by consumers as a signal that a personal care product should be changed. The detection of the odor depends, however, on the acuity of the sense of smell of the consumer, an acuity that often declines with age. Relying on the odor of the product also means that the odor must become offensive before the product is changed, an unacceptable signal.

Many technologies have been evaluated in an attempt to reduce the odors that emanate from these products during use. For example, an activated carbon ink printed liner for incontinence pads was recently introduced. Many urine odor ranking panel (ORP) studies have shown improvements in odor for the carbon treated liner. However, complete elimination of odor has not been achieved with activated carbon.

It is also important that anything added to a personal care product to reduce odor should remain in place and not migrate through the product, as has occurred in previous attempts to address this matter. Absorbent and/or adsorbent (sorbent) particles, for example, should not escape from the product nor be noticeable to the consumer.

It is clear that there exists a need for a process and product that allow for the control of odors due to bodily wastes in personal care products.

### SUMMARY

The work disclosed herein has identified a fabric treatment combination that provides urine insulted incontinence pads with the same total odor intensity ranking as pads that are insulted with only water. This fabric treatment combo can be easily incorporated into disposable incontinent products as described below.

In response to the foregoing difficulties encountered by those of skill in the art, a dual element odor control layer for personal care products includes a dried, fluid-deposited formulation of odor sorbent in the form of activated carbon and a dried, liquid-deposited formulation of a silver-based substance. This layer can be placed in a personal care product like diapers, training pants, absorbent underpants, adult incontinence products, and feminine hygiene products. The layer can be a tissue, film, paper towel, nonwoven web, coform, airlaid, wet-laid, bonded-carded web and laminates thereof.

The present disclosure includes feminine hygiene products and adult incontinence products having a liquid impermeable baffle, a liquid pervious body side liner, and a substrate having thereon a dried, aqueously or non-aqueously applied layer of odor sorbent and a silver-based substance.

A personal care product having the odor-reduction treatment of the present disclosure has odor reduction superior to a similar product lacking such an odor-reduction treatment.

An odor control layer for personal care products includes a first portion with a formulation of activated carbon and a second portion with a formulation of nanoparticle silver. The layer can be disposed in a personal care product such as diapers, training pants, absorbent underpants, adult incontinence products, and feminine hygiene products.

A personal care product includes an odor control layer including a first portion with a formulation of activated carbon and a second portion with a formulation of nanoparticle silver. The personal care product can be diapers, training pants, absorbent underpants, adult incontinence products, or feminine hygiene products.

A personal care product can include a liquid-permeable bodyside liner, a liquid-impermeable outer cover affixed to the bodyside line, and an absorbent core disposed between the bodyside liner and the outer cover. The personal care product can also include an odor control layer including a first portion with a formulation of activated carbon and a second portion with a formulation of nanoparticle silver. The personal care product can be diapers, training pants, absorbent underpants, adult incontinence products, or feminine hygiene products.

### BRIEF DESCRIPTION OF THE DRAWINGS

The present disclosure will be more fully understood, and further features will become apparent, when reference is made to the following detailed description and the accompanying drawings. The drawings are merely representative and are not intended to limit the scope of the claims.

FIG. 1 is a drawing of a feminine hygiene product.

FIG. 2 is a drawing of an adult incontinence product.

FIG. 3 is a drawing of a cross-section of an adult incontinence product.

FIG. 4 is a drawing of an absorbent underpant.

FIG. 5 is a schematic representation of an example of a substrate including the odor elimination feature described herein.

FIG. 6 is a chart illustrating the results of odor testing with different odor eliminating substances.

FIG. 7 is a chart illustrating the results of odor testing with different odor eliminating substances.

FIG. 8 is a chart illustrating the results of odor testing with different odor eliminating substances.

FIG. 9 is a chart illustrating the results of odor testing with different odor eliminating substances.

FIG. 10 is a chart illustrating the results of odor testing with different odor eliminating substances.

FIG. 11 is a chart illustrating the results of odor testing with different odor eliminating substances.

Repeat use of reference characters in the present specification and drawings is intended to represent the same or analogous features or elements of the present disclosure. The drawings are representational and are not necessarily drawn to scale. Certain proportions thereof might be exaggerated, while others might be minimized.

### DETAILED DESCRIPTION

The present disclosure involves the control of odors in personal care products, e.g., diapers, training pants, absorbent underpants, adult incontinence products, and feminine hygiene products.

The control of odors in personal care products is of particular interest to adults like those who wear feminine hygiene pads and incontinence products. The desire to avoid embarrassment due to unpleasant odors is important to adult consumers of these products and the present disclosure helps greatly in this regard.

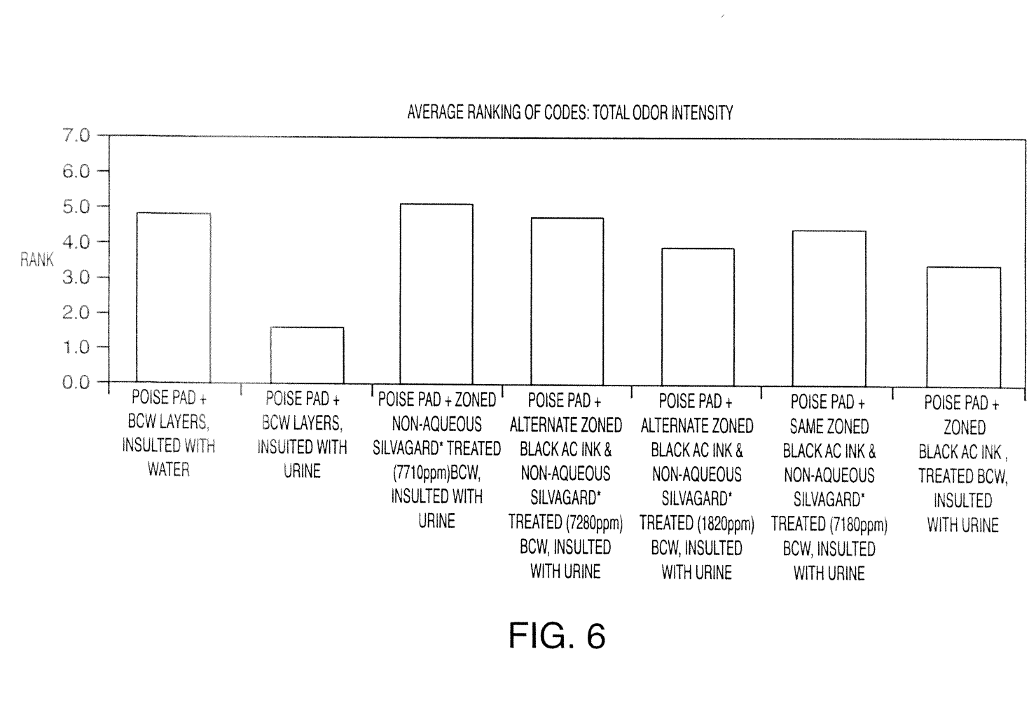

The treatment combination for odor elimination involves two principle components: activated carbon and silver. Silver is well known as an antimicrobial agent and activated carbon is known for adsorption of organic volatiles, including some of the odors that are released from urine-insulted personal care products. Silver can be deposited on many types of carriers to control the release properties of silver ions. For example, powders such as activated carbon, silica, zeolites, alumina, etc. have been used as carriers for silver. The present disclosure does not use these prior approaches of combining silver with activated carbon as a carrier. Instead, these two principle components are placed in separate locations or as separate applications in the disposable product to accommodate ease of manufacture, unfettered access to each odor elimination substance, and in some aspects the elimination of a need for separate substrates. For example, FIG. 6 illustrates the results of testing a sample having the same-zoned black activated carbon ink and non-aqueous SILVAGARD silver nanoparticles (described in more detail below) in which the silver nanoparticles are disposed on top of the activated carbon such that both components occupy the same zone on the substrate.

This odor elimination feature can be deposited using a number of methods and remains substantially in place despite the rigors of product use.

The odor sorbent can be zeolites, silicas, aluminas, titanias, sodium carbonates, sodium bicarbonates, sodium phosphates, zinc and copper sulfates, and activated carbon in particle or fiber form, or other chemicals known to control odors, and mixtures thereof. The odor sorbent is preferably activated carbon.

Examples of formulations available that contain sorbents include Nuchar PMA Ink from MeadWestvaco Corporation of New York, N.Y., USA. Other sorbent products are available from the Calgon Carbon Corporation of Pittsburgh, Pa., USA, under the trade name CARBABSORB, from Sigma-Aldrich Chemical Company of Milwaukee, Wis., USA and from Cabot Corporation of Boston, Mass., USA.

The unique nature of the activated carbon preferably used herein results from the small size of the particles. In one example, black ink is prepared including activated carbon particles up to 10 microns in diameter, preferably up to 5 microns in diameter, more preferably up to 2 microns in diameter, and even more preferably up to 1 micron in diameter. Ink including activated carbon particles can also be colored ink, such as those described in U.S. Pat. No. 7,531,471 to Quincy, III, which is incorporated herein by reference to the extent it does not conflict herewith.

For the inks used in the examples below, the black ink included activated carbon particles of approximately 1 micron in diameter, whereas the blue ink included activated carbon particles of approximately 20 microns in diameter.

Improved odor adsorption with the black ink resulted from the amount of activated carbon particles in the ink. For example, the blue ink's inclusion of larger activated carbon particles 20 microns in diameter or larger resulted in fewer activated carbon particles due to the need to prevent settling of the particles, which is more of a challenge with larger particles.

The use of activated carbon ink in absorbent articles is described in further detail in U.S. Pat. No. 7,816,285 to MacDonald et al. and in U.S. Pat. No. 7,655,829 to MacDonald et al., each of which is incorporated herein by reference to the extent it does not conflict herewith.

The silver formulation is preferably SILVAGARD silver nanoparticles available from AcryMed, Inc. of Beaverton, Oreg. USA. According to AcryMed, Inc., SILVAGARD silver nanoparticles are formed chemically in a solution. SILVAGARD silver nanoparticles can also be referred to herein as SilvaGard®, SilvaGard*, SilvaGard, or Silverguard. The nanoparticles are uniform in size (about 10 nm) and because of proprietary technology they do not agglomerate to form large particles, but stay in suspension pending application to other materials. After the SILVAGARD silver nanoparticle solution is prepared, the substrate is contacted with the solution by immersing, spraying, printing, or by any other suitable application means. The amount of nanoparticle silver actually deposited is controlled by adjusting the silver concentration and the temperature of the solution as well as the dwell time in contact with the solution. The SILVAGARD silver nanoparticle technology is further described in U.S. Patent Application Publication No. 2007/003603A1 to Karandikar et al., which is incorporated herein by reference to the extent it does not conflict herewith. Examples B7 and B14 disclosed therein are of particular usefulness in the present application. Thereafter, the resulting solution is purified to remove all small molecules (inorganic and organic) by suitable techniques known to those of skill in the art.

Because of their size, the nanoparticles attach to the surface being treated while the substrate is in contact with the SILVAGARD silver nanoparticle solution. The attachment is very uniform over the surface of the substrate. Once treated, the substrate is removed and dried. The SILVAGARD silver nanoparticles adhere tenaciously even on elastic substrates when they are stretched or flexed.

The solution treatment process can be either aqueous or solvent based, depending on the needs or characteristics of the substrate to be treated. Each nanoparticle would theoretically contain a small number (approximately 5,000) of silver atoms. The outer layer of silver in each particle oxidizes upon exposure to air or bodily fluid. This process forms a monolayer of Ag₂O (silver oxide) on the outside of the nanoparticle. The silver oxide then slowly dissolves when contacted by the body fluid it encounters on the substrate. It dissolves to produce Ag+, ionic silver.

It should be noted that the SILVAGARD silver nanoparticles are applied to the substrate without the use of a binder. Silver components in dry form made into a suspension with a binder and applied to a substrate do not provide the same effect because the surfaces of the silver components can be shielded by the binder. In the present application, the combination of the binder-less application of the SILVAGARD silver nanoparticles along with non-agglomerative nature of the SILVAGARD silver nanoparticles allows for the maximum surface area available to interact with odor-causing agents. While the specific mechanisms of this interaction can be multiple and complex, it is thought that the odor-eliminating effectiveness of the SILVAGARD silver nanoparticles is due in large part to the adsorption of odor-causing agents.

The vast number of SILVAGARD silver nanoparticles on the surface of the substrate provides a very large reservoir and surface area of silver for continuous protection. It is this very large surface area of silver that gives SILVAGARD silver nanoparticles effectiveness at very low concentrations, very low cytotoxicity, and long lasting, sustained release.

The combination of odor-eliminating components described herein has been found to have a synergistic effect in eliminating odors. The combination provides a significantly larger odor reduction that can be achieved by either component alone, even at higher concentrations. If other forms of silver (e.g., silver/zeolite) or even other forms of silver nanoparticles (e.g., silver nanoparticle dispersion stabilized by a binder) are used, the synergistic effect is largely absent.

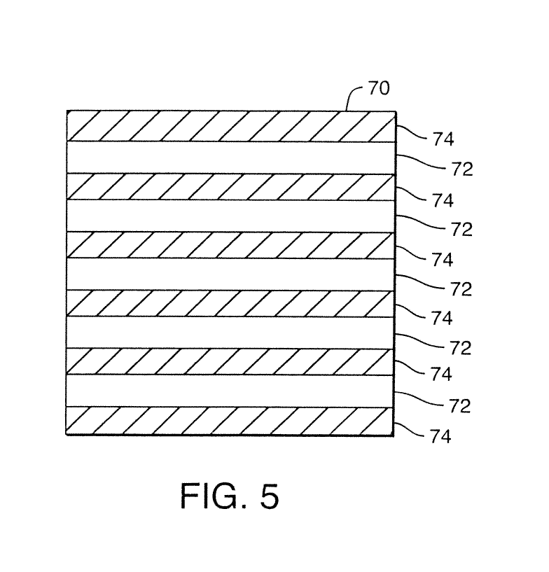

Separate formulations of activated carbon and silver nanoparticles are applied to a nonwoven substrate 70 (see FIG. 5). The nonwoven substrate 70 can be a bonded carded web (BCW), or any other suitable nonwoven or other substrate described herein. In a particular aspect, the BCW is made with two types of fibers; a 3 denier bicomponent fiber with a polyethylene sheath and a polypropylene core and a 6 denier polyester fiber, with a ratio of bicomponent fibers to polyester fibers of 3 to 1. The silver component 72 can be applied to the nonwoven substrate 70 by contacting the nonwoven substrate 70 with a non-aqueous liquid formulation containing silver nanoparticles and heptane, or with an aqueous silver nanoparticle solution, followed by air drying. The dried nonwoven substrate 70 then includes silver nanoparticles attached to the nonwoven substrate fibers. This silver treatment is the SILVAGARD silver nanoparticle process described above. The activated carbon component 74 can be applied to the nonwoven substrate 70 by spraying an activated carbon ink formulation to zones of the nonwoven substrate 70. The zone-sprayed nonwoven substrate 70 is then air dried. In one aspect, a template or mask can be placed over the nonwoven substrate 70 prior to spraying to allow the spray to produce the zones of activated carbon treatment.

The activated carbon component 74 and the silver component 72 can both be applied to the same side of the nonwoven substrate 70, can both be applied to both sides of the nonwoven substrate 70, or the activated carbon component 74 can be applied to one side of the nonwoven substrate 70, with the silver component 72 applied to the other side of the nonwoven substrate 70. Each activated carbon component 74 of the nonwoven substrate 70 can be separate from each silver component 72 of the nonwoven substrate 70, or the activated carbon component 74 can overlap the silver component 72. The activated carbon component 74 and the silver component 72 can form alternating stripes (see FIG. 5) or any other shapes or patterns.

Personal care products that include the nonwoven substrate 70 including the odor elimination feature described herein can include diapers, training pants, feminine hygiene products, incontinence products, and absorbent underpants.

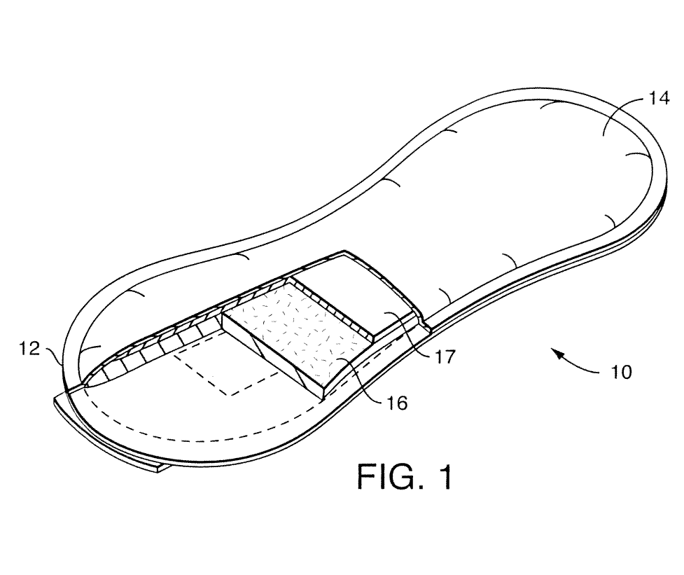

A nonwoven substrate 70 including the odor elimination feature described herein can be included in feminine hygiene products as mentioned above. These include, for example, the pad shown partially cut away in FIG. 1. This pad 10 has a liquid impermeable baffle or outer cover 12 on the side away from the wearer. The baffle 12 is often made from a film like a polyethylene or polypropylene film. The layer closest to the wearer is the liner 14 and is a liquid permeable layer that is preferably soft. Between the baffle 12 and liner 14 there can be a number of layers for different purposes, such as an absorbent core 16 designed to hold the majority of any liquid discharge. Other optional layers include a transfer delay layer or surge layer 17, and tissue or nonwoven wrap sheets (not shown).

Disposable absorbent incontinence products are designed to be removed and discarded after a single use. By single use it is meant that the disposable absorbent incontinence product will be disposed of after being used once instead of being laundered or cleaned for reuse, as is typical of regular cloth underwear. Examples of some commercially available disposable absorbent incontinence products include diapers, training pants, pads, pantiliners, fitted briefs, belted shields, guards for men, protective underwear, and adjustable underwear.

Many of the disposable absorbent incontinence underwear are similar in appearance, size, and shape to regular cloth underwear except that they are formed from a variety of different materials including absorbent and elastic materials. The absorbent materials allow the disposable absorbent incontinence underwear to absorb and retain body waste while the elastic material permits the disposable absorbent incontinence underwear to snugly conform to the anatomy of the wearer's torso.

Much of the disposable absorbent incontinence underwear sold today has a unitary configuration that is similar to regular cloth underwear in that the disposable absorbent incontinence underwear is constructed with a waist opening and a pair of leg openings and needs to be pulled onto the body like normal underwear. Another aspect of unitary disposable absorbent underwear is disclosed in U.S. Patent Publication No. 2004/0210205 A1 to Van Compel et al., which is incorporated herein in its entirety by reference thereto to the extent it does not conflict herewith.

Other disposable absorbent incontinence underwear has an open configuration. By an open configuration it is meant that the disposable absorbent incontinence underwear does not have a waist opening and a pair of leg openings before it is positioned about the wearer's torso. Typically, disposable absorbent incontinence underwear having an open configuration has a relatively flat or convex shape before it is secured around the torso of the wearer. Commonly, disposable absorbent incontinence underwear having an open configuration has an approximately rectangular or hourglass shape. Such products are described in U.S. Pat. No. 4,500,316 to Damico, which is incorporated herein in its entirety by reference thereto to the extent it does not conflict herewith.

An adjustable undergarment, also sometimes referred to as refastenable underwear, has a unitary configuration and can be positioned onto the wearer's body similar to regular cloth underwear. However, the adjustable undergarment has the ability to be opened and then refastened into a closed position during use.

As stated above, disposable absorbent incontinence products are manufactured in a variety of shapes and configurations. Another type of incontinence product is a guard for men, which resembles an absorbent pad that can conform to the male genitalia, as described in U.S. Pat. No. 5,558,659 to Sherrod et al., which is incorporated herein in its entirety by reference thereto to the extent it does not conflict herewith. A belted shield is still another type of a disposable absorbent incontinence product that has an open configuration and is held about the wearer's torso by a belt or a pair of straps, as described in U.S. Pat. No. 5,386,595 to Kuen et al. and U.S. Pat. No. 4,886,512 to Damico et al., which are incorporated herein in their entirety by reference thereto to the extent they do not conflict herewith.

Female incontinence consumers can use various incontinence products similar to or as a variation of those described above, including pads, pantiliners, fitted briefs, belted shields, protective underwear and adjustable underwear.

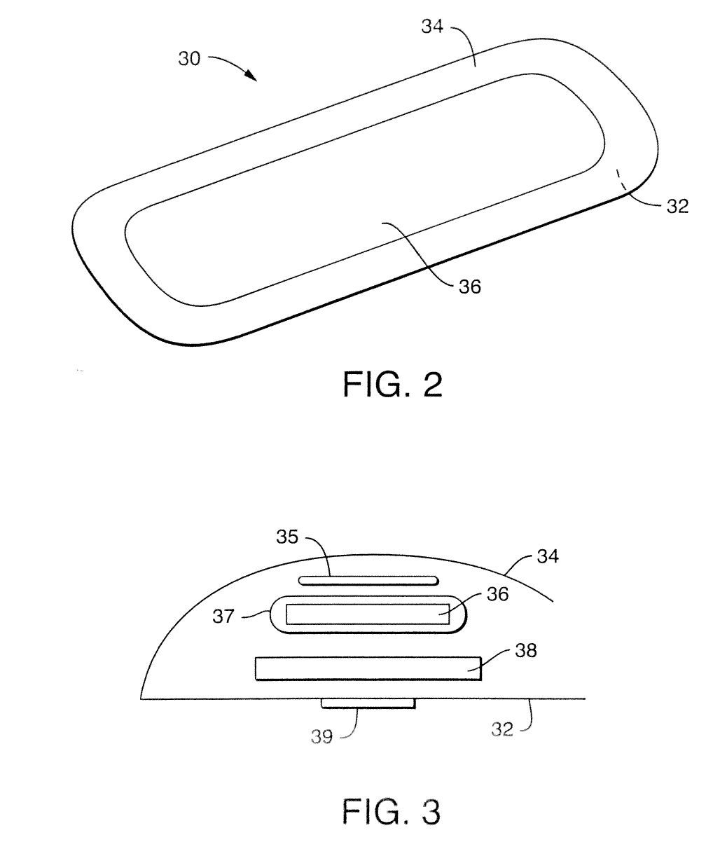

Incontinence pads 30 as shown in FIG. 2 likewise have a baffle or outer cover 32, an innermost liner 34, and various layers in between, including the absorbent core 36. FIG. 3 illustrates an incontinence product in cross-section where the section is taken across the narrow part of the product. The liner 34 is at the top, and a surge layer 35 is positioned below the liner 34. The surge layer 35 acts as a reservoir to accept large surges of liquid and slowly release them to the subsequent layers. Below the surge layer 35 is an absorbent core or pledget 36 surrounded by tissue wrap 37. The absorbent core 36 can include superabsorbent particles that are loose and very small and that can escape onto the body or clothing unless contained. The tissue wrap 37 surrounds the absorbent core 36 and keeps the superabsorbent particles from leaving the absorbent core 36. Under the tissue wrapped absorbent core 36 is a fluff layer 38 and then the baffle 32. Many products also have an adhesive strip 39 to help hold the product in place during use by adhering it to the user's underclothes. More information concerning incontinence products can be found, for example, in U.S. Pat. No. 6,921,393 to Tears et al., which is incorporated herein in its entirety by reference thereto to the extent it does not conflict herewith.

The liquid permeable liner 34 is designed to allow body fluid, particularly urine, to quickly pass therethrough and be received by the absorbent core 36. The bodyside liner 34 is placed in contact with the genital area of a human body. The bodyside liner 34 is capable of passing body fluid, voluntarily or involuntarily expelled from the urethra, downward into the absorbent core 36.

Pads typically have an approximately rectangular, hourglass, or asymmetrical configuration having a thickness of about 2.5 centimeters (cm) or less. Desirably, the thickness of a pad is less than about 1 cm. More desirably, the thickness of a pad is less than about 0.7 cm. A pad can have a length of from between about 15 cm to about 50 cm and a width of from between about 2 cm to about 15 cm.

A pantiliner is another female incontinence product. By pantiliner it is meant a thin absorbent product having an approximately rectangular, hourglass or asymmetrical configuration having a thickness of about 1 cm or less. Desirably, the thickness of a pantiliner is less than about 0.9 cm. More desirably, the thickness of a pantiliner is less than about 0.5 cm. A pantiliner can have a length of from between about 15 cm to about 50 cm and a width of from between about 2 cm to about 15 cm.

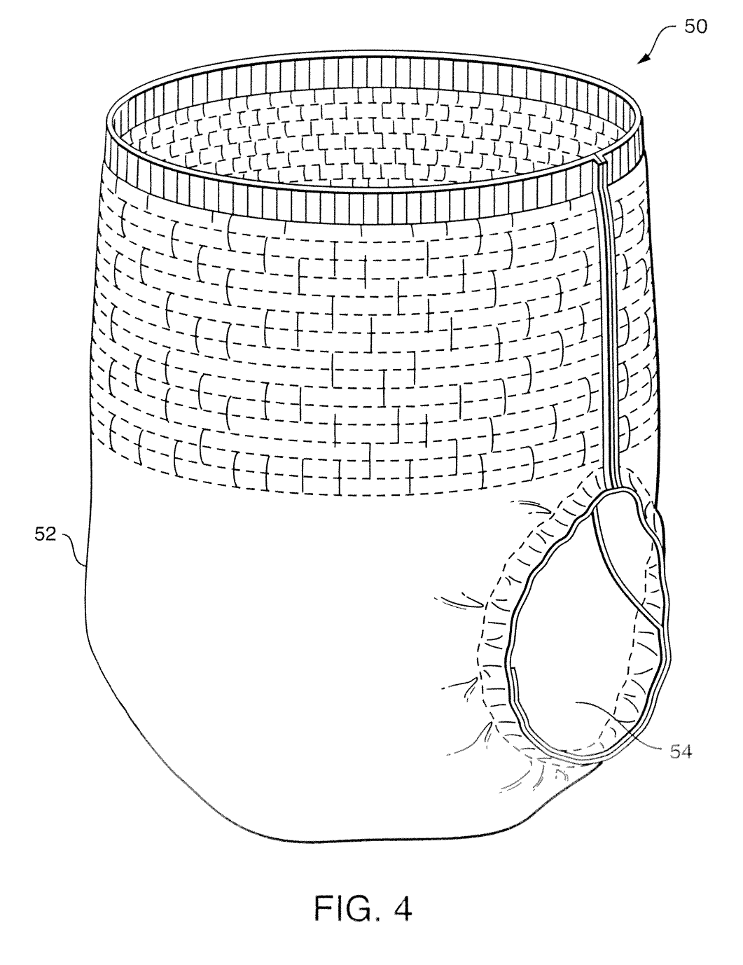

Absorbent underpants 50 as shown in FIG. 4 have a baffle 52, a liner 54, and an absorbent core (not shown). Further discussion regarding absorbent underpants can be found, for example, in U.S. Pat. No. 6,240,569 to Van Gompel and in U.S. Pat. No. 6,367,089 to Van Gompel, which are incorporated herein in their entirety by reference thereto to the extent they do not conflict herewith.

The substrate including the odor elimination feature described herein can be included in any of the personal care products described herein as an additional layer to those described, or in the place of a layer described herein.

### EXAMPLES

### Example Set 1

Separate formulations of activated carbon and silver were applied to a single nonwoven substrate 70 (FIG. 5). The nonwoven substrate 70 was a bonded carded web (BCW) with a basis weight of 0.9 osy.

The activated carbon component was applied to the BCW by using a mask in the form of a grill grate (stainless steel gas grill grate model 9869 from Weber-Stephens Products Co., Palatine, Ill.) to form a pattern of stripes on the BCW, and then spraying the masked BCW with an activated carbon ink, followed by air drying. The silver component was applied to the BCW by repositioning the same mask to form a pattern of complementary stripes on the BCW, and then spraying the masked BCW with a liquid formulation containing silver nanoparticles and heptane, followed by air drying. The dried BCW contained silver nanoparticles attached to the BCW fibers. This silver treatment is the SILVAGARD silver nanoparticle treatment described above. A schematic representation of the zones employed is illustrated in FIG. 5, wherein first portions 74 are the portions of the nonwoven substrate 70 treated with activated carbon ink, and the second portions 72 are the portions treated with silver. The amount of silver applied is listed in Table 1.

TABLE 1

| | Sample | Silver Conc | mg Ag/ | Avg | |
|---|---|---|---|---|---|
| Sample | wt (mg) | (wt %) | sample | mg Ag/cm² | ppm |
| 1 | 145.74 | 0.771 | 1.124 | 0.0249 | 7710 |
| 2 | 172.33 | 0.728 | 1.255 | 0.0278 | 7280 |
| 3 | 179.55 | 0.718 | 1.289 | 0.0285 | 7180 |
| 4 | 185.30 | 0.182 | 0.337 | 0.0075 | 1820 |
| 5 | 122.13 | 0.457 | 0.558 | 0.0124 | 4570 |

Each sample was 2.5 cm×17.8 cm for a sample area of 45.16 cm²

POISE incontinence pads available from Kimberly-Clark Corp. of Dallas, Tex. USA with the label “Moderate Absorbency” were purchased from a local store for a urine odor ranking panel (ORP) study. The surge layer was removed from each pad to make space to accommodate the treated 0.9 osy BCW fabrics. Two pieces of BCW were inserted with the top layer always being a piece cut from the original roll and designated as untreated BCW layer. The bottom layer contained the various treatments described in Table 2. After inserting the two pieces of BCW, the top liner was pulled back into place and one staple was applied at the middle edge of the pad to hold the contents in place. Table 2 describes the codes that were tested in the urine ORP study.

TABLE 2

| Code ID | Codes | Components |
|---|---|---|
| 912 | POISE control (water insult) | POISE pad less surge layer + two BCW layers, |
| | | insulted with water |
| 197 | POISE control (urine insult) | POISE pad less surge layer + two BCW layers, |
| | | insulted with urine |
| 486 | SILVAGARD silver nanoparticles | POISE pad less surge layer + zoned non-aqueous |
| | | SILVAGARD silver nanoparticles-treated (7710 ppm) |
| | | BCW + one BCW layer |
| 875 | AC Ink - Black + SILVAGARD | POISE pad less surge layer + alternate zoned Black |
| | silver nanoparticles | AC Ink/non-aqueous SILVAGARD silver nanoparticles- |
| | | treated (7280 ppm) BCW + one BCW layer |
| 604 | AC Ink - Black + SILVAGARD | POISE pad less surge layer + alternate zoned Black |
| | silver nanoparticles | AC Ink/non-aqueous SILVAGARD silver nanoparticles- |
| | | treated (1820 ppm) BCW + one BCW layer |
| 243 | AC Ink - Black + SILVAGARD | POISE pad less surge layer + overlapping zoned Black |
| | silver nanoparticles | AC Ink/non-aqueous SILVAGARD silver nanoparticles- |
| | | treated (7180 ppm) BCW + one BCW layer |
| 326 | AC Ink - Black | POISE pad less surge layer + zoned Black AC Ink |
| | | BCW + one BCW layer |

There were 10 pads per code evaluated in the Odor Ranking Panel.

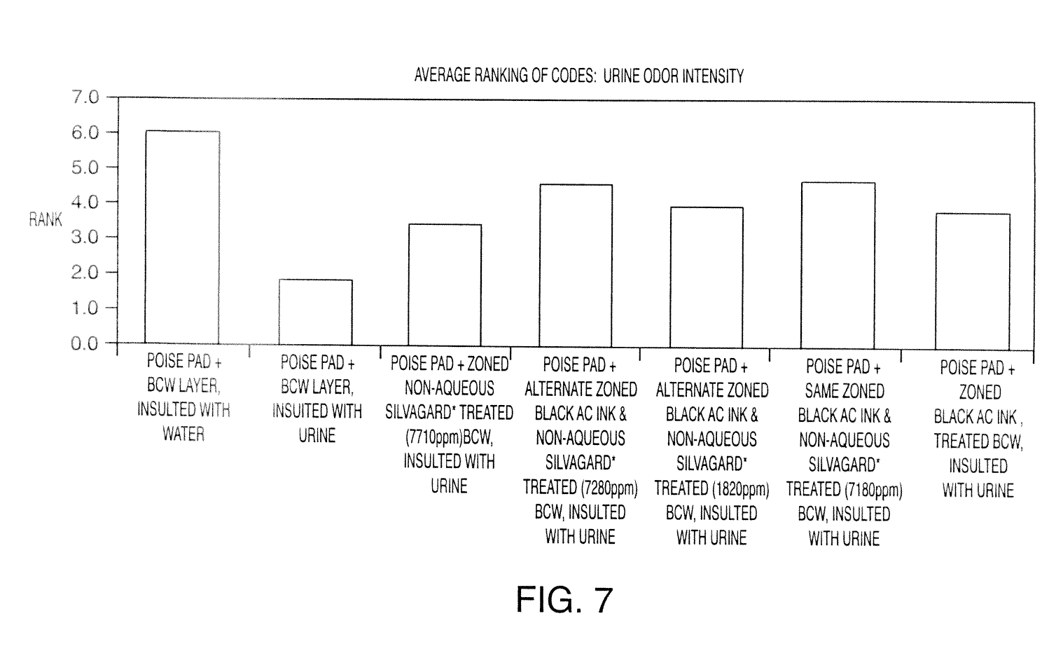

Human urine was collected, pooled, filter sterilized, and then inoculated with bacteria (Proteus mirabilis, Klebsiella pnuemonae, E. faecalis, and E. coli). A fixed amount of urine (78 ml) was placed on each pad and the pad was incubated at 37° C. for four hours. Ten panelists were then exposed to each of the seven codes and asked to rank them for overall odor and for urine odor. The results are shown in FIGS. 6 and 7 for overall odor and for urine odor, respectively, in which the controls were the pad insulted with urine and the pad insulted with water. Codes with rankings above the urine control result were judged to produce fewer odors. It can be seen that activated carbon ink plus SILVAGARD silver nanoparticles is effective for both total odor and urine odor, while the SILVAGARD silver nanoparticles alone were effective only for the total odor. A synergistic effect was achieved by having activated carbon and silver nanoparticles together. The components together yielded a significantly larger odor reduction than could be achieved by either technology by itself, even at a higher add-on.

### Example Set 2

Separate formulations of activated carbon and silver were applied to nonwoven substrates 70 (FIG. 5). Each nonwoven substrate 70 was a bonded carded web (BCW) with a basis weight of 0.9 osy. The BCW was made with two types of fibers: a 3 denier bicomponent fiber with a polyethylene sheath and a polypropylene core and a 6 denier polyester fiber. The ratio of bicomponent fibers to polyester fibers was 3 to 1.

The silver component was applied to the BCW by soaking it in a liquid formulation containing silver nanoparticles and heptane, followed by air drying. The dried BCW contained silver nanoparticles attached to the BCW fibers. This silver treatment is the SILVAGARD silver nanoparticle treatment described above. The activated carbon component was applied to another BCW by using a grill to mask stripes on the BCW, and then spraying the masked BCW with an activated carbon ink formulation. The zone-sprayed BCW was then air dried. Two different activated carbon ink formulations were used. They are designated as “AC Ink—Black” and “AC Ink—Blue” (described above).

POISE incontinence pads available from Kimberly-Clark Corp. of Dallas, Tex. with the label “Moderate Absorbency” were purchased from a local store for a urine odor ranking panel (ORP) study. The surge layer was removed from each pad to make space to accommodate the treated 0.9 osy BCW fabrics. Table 3 describes the codes that were tested in this urine ORP study. The amount of the various treatments is shown in Table 4 for each code.

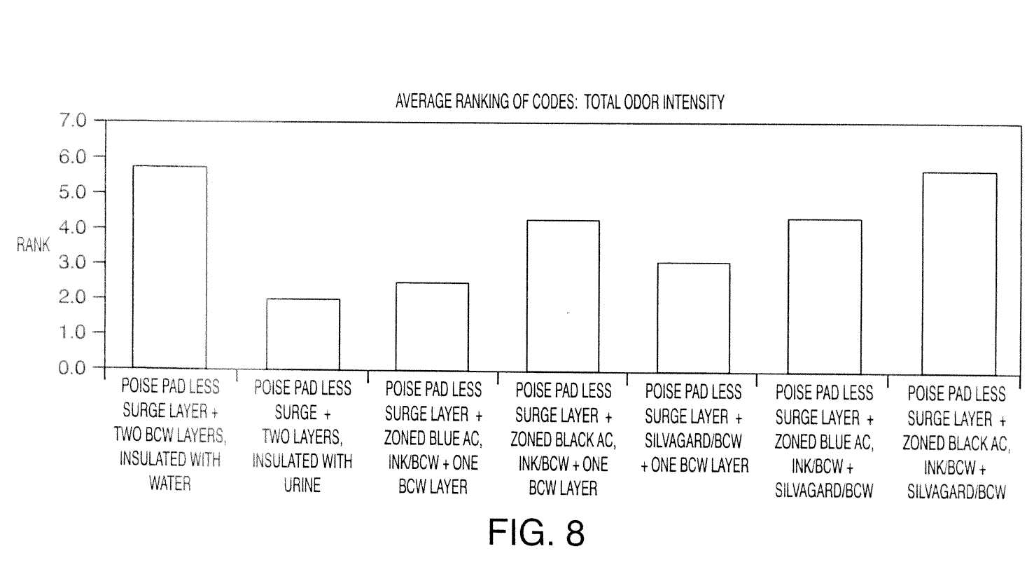

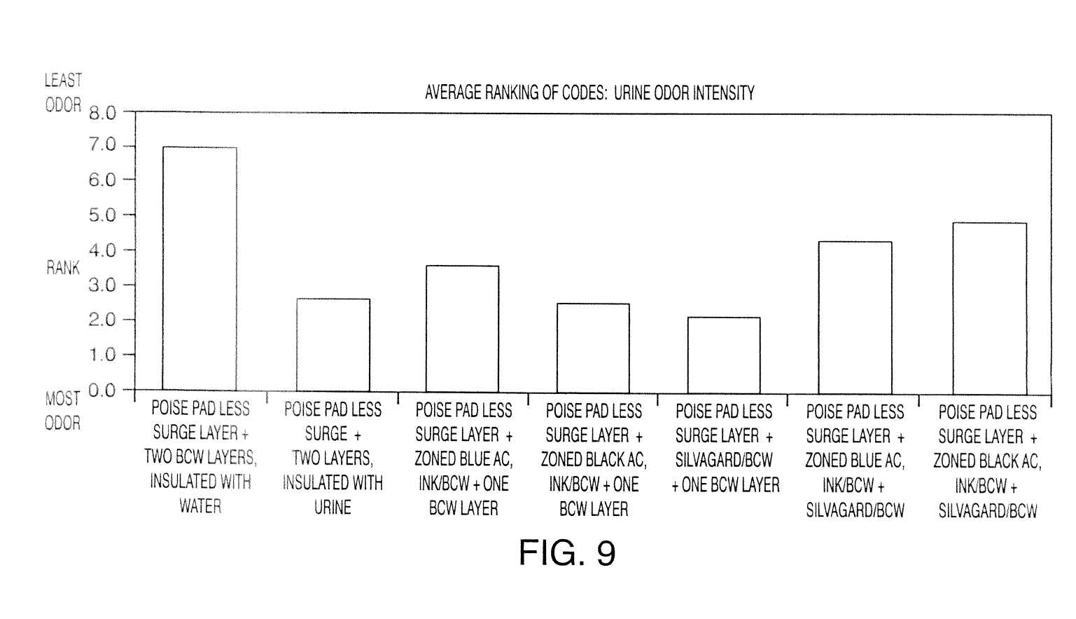

Human urine was collected, pooled, filter sterilized, and then inoculated with bacteria (Proteus mirabilis, Klebsiella pnuemonae, E. faecalis, and E. coli). A fixed amount of urine (78 ml) was placed on each pad and the pad was incubated at 37° C. for four hours. Ten panelists were then exposed to each of the seven codes and asked to rank them for overall odor. The results are shown in FIGS. 8 and 9, in which the controls were the pad insulted with urine and the pad insulted with water. Codes with rankings above the urine control were judged to produce fewer odors. It can be seen that the code with AC Ink—Black and SILVAGARD silver nanoparticles performed the best. In fact, the ranking for total odor was the same as the control pad that was insulted with only water. A synergistic effect was achieved by having activated carbon and silver nanoparticles together. The components together yielded a significantly larger odor reduction than could be achieved by either technology by itself, even at a higher add-on.

TABLE 3

| Code ID | Codes | Components |
|---|---|---|
| 126 | POISE control (water insult) | POISE pad less surge layer + two BCW layers, |
| | | insulted with water |
| 680 | POISE control (urine insult) | POISE pad less surge layer + two BCW layers, |
| | | insulted with urine |
| 473 | Activated Carbon Ink - Blue | POISE pad less surge layer + zoned Blue AC |
| | | Ink/BCW + one BCW layer |
| 719 | Activated Carbon Ink - Black | POISE pad less surge layer + zoned Black AC |
| | | Ink/BCW + one BCW layer |
| 226 | SILVAGARD silver nanoparticles | POISE pad less surge layer + SILVAGARD silver |
| | | nanoparticles/BCW + one BCW layer |
| 845 | AC Ink - Blue + SILVAGARD | POISE pad less surge layer + zoned Blue AC |
| | silver nanoparticles | Ink/BCW + SILVAGARD silver nanopartieles/BCW |
| 531 | AC Ink - Black + SILVAGARD | POISE pad less surge layer + zoned Black AC |
| | silver nanoparticles | Ink/BCW + SILVAGARD silver nanoparticles/BCW |

All treatments were applied to 0.9 osy (30.5 gsm) BCW. The treated BCW pieces were inserted into a POISE pad after removing the existing surge layer from the pad. The pieces were cut to 1″ MD by 7″ CD in size for the zoned Black AC Ink/BCW and the SILVAGARD silver nanoparticles/BCW and to 2″ MD by 7″ CD for the other treated pieces. There were 10 pads per code evaluated in the Odor Ranking Panel.

TABLE 4

| | | | | Size & Number | |
|---|---|---|---|---|---|
| | | Amount on | Amount on | of pieces in | Amount in POISE |
| Code ID | Treatment | BCW (%) | BCW (gsm) | POISE Pad | Pad (mg) |
| 473 | AC Ink - Blue | Total Ink | Total Ink | One 2″ × 7″ | 22.9 |
| | | Solids: 8.30% | Solids: 2.53 | piece | |
| | | Activated | Activated | | 6.6 |
| | | Carbon: 2.39% | Carbon: 0.73 | | |
| 719 | AC Ink - Black | Total Ink | Total Ink | One 1″ × 7″ | 25.3 |
| | | Solids: 18.4% | Solids: 5.6 | piece | |
| | | Activated | Activated | | 14.5 |
| | | Carbon: 10.4% | Carbon: 3.2 | | |
| 226 | SILVAGARD silver | Amount of | Amount of | One 1″ × 7″ | 1.5 |
| | nanoparticles | Silver: 1.12% | Silver: 0.34 | piece | |
| 845 | AC Ink - Blue | Total Ink | Total Ink | One 2″ × 7″ | 22.9 |
| | | Solids: 8.30% | Solids: 2.53 | piece | |
| | | Activated | Activated | | 6.6 |
| | | Carbon: 2.39% | Carbon: 0.73 | | |
| | SILVAGARD silver | Amount of | Amount of | One 1″ × 7″ | 1.5 |
| | nanoparticles | Silver: 1.12% | Silver: 0.34 | piece | |
| 531 | AC Ink - Black | Total Ink | Total Ink | One 1″ × 7″ | 25.3 |
| | | Solids: 18.4% | Solids: 5.6 | piece | |
| | | Activated | Activated | | 14.5 |
| | | Carbon: 10.4% | Carbon: 3.2 | | |
| | SILVAGARD silver | Amount of | Amount of | One 1″ × 7″ | 1.5 |
| | nanoparticles | Silver: 1.12% | Silver: 0.34 | piece | |

Odor control occurs whether the two treatments are on separate pieces or on the same piece.

### Example Set 3

Separate formulations of activated carbon and silver were applied to a single nonwoven substrate 70. The nonwoven substrate 70 was a bonded carded web (BCW) with a basis weight of 0.9 osy.

The activated carbon component was applied to the BCW by using a mask in the form of a grill grate (stainless steel gas grill grate model 9869 from Weber-Stephens Products Co., Palatine, Ill. USA) to form a pattern of stripes on the BCW, and then spraying the masked BCW with an activated carbon ink, followed by air drying. The silver component was applied to the BCW by repositioning the same mask to form a pattern of complementary stripes on the BCW, and then spraying the masked BCW with a liquid formulation containing silver nanoparticles and heptane, followed by air drying. The dried BCW contained silver nanoparticles attached to the BCW fibers. This silver treatment is the SILVAGARD silver nanoparticle treatment described above. A schematic representation of the zones employed is illustrated in FIG. 5, wherein first portions 74 are the portions of the nonwoven substrate 70 treated with activated carbon ink, and the second portions 72 are the portions treated with silver. The amount of silver applied is listed in Table 5.

TABLE 5

| | Sample | Silver Conc | μg Ag/ |
|---|---|---|---|
| Sample | wt(mg) | (ppm) | sample |
| 1 | 126.17 | 363 | 45.80 |
| 2 | 131.74 | 473 | 62.31 |
| 3 | 196.14 | 231 | 45.31 |
| 4 | 199.14 | 275 | 54.76 |
| 5 | 178.18 | 216 | 38.49 |

Each sample was 2.5 cm×17.8 cm for a sample area of 45.16 cm²

POISE incontinence pads available from Kimberly-Clark Corp. of Dallas, Tex. USA with the label “Moderate Absorbency” were purchased from a local store for a urine odor ranking panel (ORP) study. The surge layer was removed from each pad to make space to accommodate the treated 0.9 osy BCW fabrics. Two pieces of BCW were inserted with the top layer always being a piece cut from the original roll and designated as untreated BCW layer. The bottom layer contained the various treatments described in Table 6. After inserting the two pieces of BCW, the top liner was pulled back into place and one staple was applied at the middle edge of the pad to hold the contents in place. Table 6 describes the codes that were tested in the urine ORP study.

TABLE 6

| Code ID | Codes | Components |
|---|---|---|
| 617 | POISE control (water insult) | POISE pad surge layer + two BCW layers, |
| | | insulted with water |
| 108 | POISE control (urine insult) | POISE pad less surge layer + two BCW layers, |
| | | insulted with urine |
| 942 | SILVAGARD silver nanoparticles | POISE pad surge layer + zoned non-aqueous |
| | | SILVAGARD silver nanoparticles-treated BCW + |
| | | one BCW layer |
| 831 | SILVAGARD silver nanoparticles | POISE pad less surge layer + zoned aqueous |
| | | SILVAGARD silver nanoparticles-treated BCW + |
| | | one BCW layer |
| 376 | AC Ink - Black + SILVAGARD | POISE pad less surge layer + alternate zoned Black |
| | silver nanoparticles | AC Ink/non-aqueous SILVAGARD silver nanoparticles- |
| | | treated BCW + one BCW layer |
| 564 | AC Ink - Black + SILVAGARD | POISE pad less surge layer + alternate zoned Black |
| | silver nanoparticles | AC Ink/aqueous SILVAGARD silver nanoparticles- |
| | | treated BCW + one BCW layer |
| 489 | AC Ink - Black+ SILVAGARD | POISE pad less surge layer + same-zoned Black AC |
| | silver nanoparticles | Ink/non-aqueous SILVAGARD silver nanoparticles- |
| | | treated BCW + one BCW layer |

There were 10 pads per code evaluated in the Odor Ranking Panel.

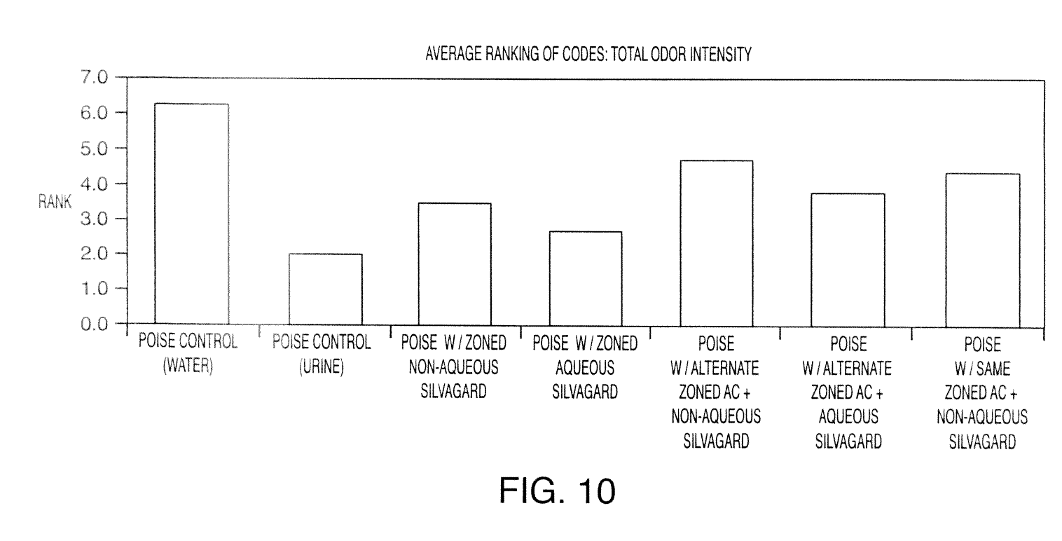

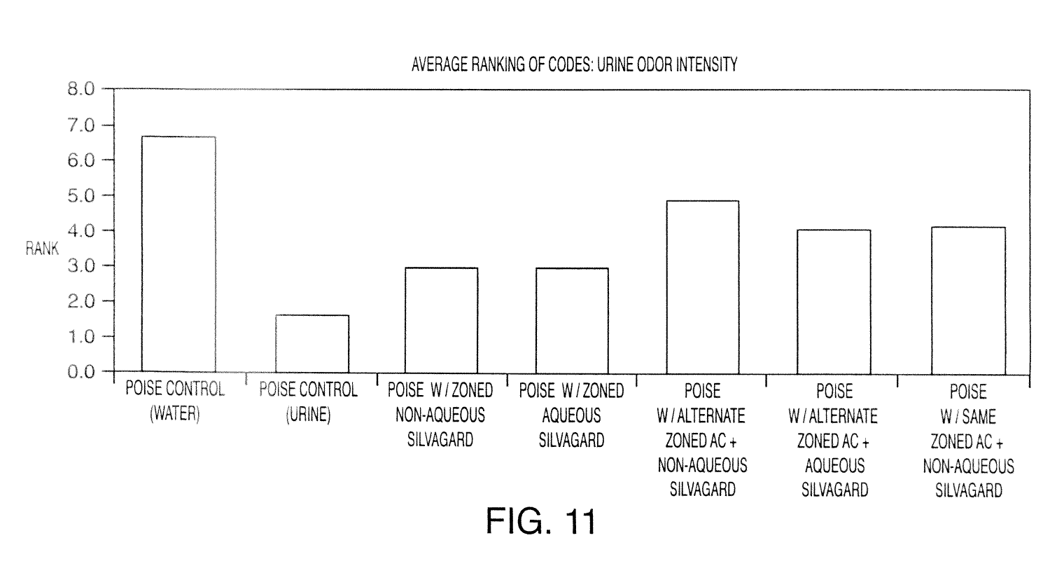

Human urine was collected, pooled, filter sterilized, and then inoculated with bacteria (Proteus mirabilis, Klebsiella pneumoniae, E. faecalis, and E. coli). A fixed amount of urine (78 ml) was placed on each pad and the pad was incubated at 37° C. for four hours. Ten panelists were then exposed to each of the seven codes and asked to rank them for overall odor. The results are shown in FIGS. 10 and 11, in which the controls were the pad insulted with urine and the pad insulted with water. Codes with rankings above the urine control were judged to produce fewer odors. It can be seen that the three codes that contained both carbon and silver performed the best for both total odor and urine odor. The reduced amounts of silver, however, in this example set yielded inferior results to those of the first two example sets.

As will be appreciated by those skilled in the art, changes and variations to the present disclosure are considered to be within the ability of those skilled in the art. Examples of such changes are contained in the patents identified above, each of which is incorporated herein by reference in its entirety to the extent it is consistent with this specification. Such changes and variations are intended by the inventors to be within the scope of the present disclosure. It is also to be understood that the scope of the present disclosure is not to be interpreted as limited to the specific aspects disclosed herein, but only in accordance with the appended claims when read in light of the foregoing disclosure.

1. An odor control layer for personal care products comprising a first portion with a formulation of activated carbon and a second portion with a formulation of nanoparticle silver, wherein the layer is disposed in a personal care product selected from the group consisting of diapers, training pants, absorbent underpants, adult incontinence products, and feminine hygiene products;
further comprising a plurality of first portions with a formulation of activated carbon and a plurality of second portions with a formulation of nanoparticle silver, the plurality of first portions and the plurality of second portions alternating across the odor control layer.

2. The odor control layer of claim 1, wherein the odor control layer further comprises a first substrate on which the activated carbon is disposed, and a second substrate on which the nanoparticle silver is disposed.

3. The odor control layer of claim 1, wherein the formulation of activated carbon is a colored, non-black activated carbon ink.

4. The odor control layer of claim 1, wherein the formulation of activated carbon is an activated carbon ink comprising activated carbon particles and a micro-cracking binder.

5. The odor control layer of claim 3, wherein the activated carbon ink includes activated carbon particles up to 10 microns in diameter.

6. The odor control layer of claim 3, wherein the activated carbon ink includes activated carbon particles up to 2 microns in diameter.

7. The odor control layer of claim 3, wherein the activated carbon ink includes activated carbon particles up to 1 micron in diameter.

8. The odor control layer of claim 1, wherein the formulation of nanoparticle silver does not include a binder.

9. The odor control layer of claim 1, wherein the odor control layer is configured to render the absorbent article when insulted with urine to have an odor equivalent to a similar article insulted with water.

10. The odor control layer of claim 1, wherein the first portion partially overlaps the second portion.

11. (canceled)

12. (canceled)

13. The odor control layer of claim 1, further comprising a plurality of first and second portions configured to produce a synergistic odor reduction benefit.

14. The odor control layer of claim 1, wherein the layer includes a first side and a second side, and wherein the first and second portions are on the first side.

15. A personal care product comprising an odor control layer including a first portion with a formulation of activated carbon and a second portion with a formulation of nanoparticle silver, wherein the personal care product is selected from the group consisting of diapers, training pants, absorbent underpants, adult incontinence products, and feminine hygiene products;
further comprising a plurality of first portions with a formulation of activated carbon and a plurality of second portions with a formulation of nanoparticle silver, the plurality of first portions and the plurality of second portions alternating across the odor control layer.

16. The personal care product of claim 15, further comprising a bodyside liner, wherein the bodyside liner includes the odor control layer.

17. The personal care product of claim 15, further comprising an outer cover, wherein the outer cover includes the odor control layer.

18. The personal care product of claim 15, further comprising an absorbent core, wherein the absorbent core includes the odor control layer.

19. A personal care product comprising:
a liquid-permeable bodyside liner;
a liquid-impermeable outer cover affixed to the bodyside liner;
an absorbent core disposed between the bodyside liner and the outer cover; and
an odor control layer including a first portion with a formulation of activated carbon and a second portion with a formulation of nanoparticle silver, wherein the personal care product is selected from the group consisting of diapers, training pants, absorbent underpants, adult incontinence products, and feminine hygiene products;
further comprising a plurality of first portions with a formulation of activated carbon and a plurality of second portions with a formulation of nanoparticle silver, the plurality of first portions and the plurality of second portions alternating across the odor control layer.

20. The personal care product of claim 19, wherein the bodyside liner includes the odor control layer.
